(19) United States
(12) Patent Application Publication
(10) Pub. No.: US 2026/0220906 A1
(43) Pub. Date: Jul. 30, 2026

(54) MEDICAL DEVICE, METHOD, AND STORAGE MEDIUM

(71) Applicant: TERUMO KABUSHIKI KAISHA, Tokyo (JP)

(72) Inventor: Yuki SAKAGUCHI, Fujisawa Kanagawa (JP)

(21) Appl. No.: 19/578,767

(22) Filed: Mar. 25, 2026

Related U.S. Application Data
(63) Continuation of application No. PCT/JP2024/033836, filed on Sep. 24, 2024.

(30) Foreign Application Priority Data
Sep. 25, 2023 (JP) 2023-161616

(51) Int. Cl. G06V 10/25; A61B 5/00; G06T 7/00; G06T 7/73; G06T 11/23; G06V 10/774; G06V 20/70

(52) U.S. Cl. CPC G06V 10/25; A61B 5/0073; A61B 5/0082; A61B 5/743; G06T 7/0012; G06T 7/73; G06T 11/23; G06V 10/774; G06V 20/70; G06T 2207/10072; G06T 2207/20081; G06T 2207/30021; G06T 2207/30101; G06T 2210/41; G06V 2201/034

(57) ABSTRACT

A medical device for processing a blood vessel tomographic image includes a display and a processor that acquires data indicating a blood vessel tomographic image and executes a call to a machine learning model to detect a plurality of target regions and an unclear part in the blood vessel tomographic image. The model is trained with training blood vessel tomographic images and ground truth labels. The processor identifies a first boundary portion of a target region among the plurality of target regions, the first boundary portion being outside the detected unclear part, and corrects a second boundary portion of the target region that is inside the detected unclear part, using the identified first boundary portion. The processor generates and controls the display to display a screen showing the blood vessel tomographic image and a superimposed boundary line including the first boundary portion and the corrected second boundary portion.

### CROSS-REFERENCE O RELATED APPLICATION

This application is a continuation of International Patent Application No. PCT/JP2024/033836 filed September 24, 2024, which is based upon and claims the benefit of priority from Japanese Patent Application No.2023-161616, filed September 25, 2023, the entire contents of which are incorporated herein by reference.

### TECHNICAL FIELD

The present disclosure relates to a medical device, a method, and a storage medium.

### BACKGROUND ART

To support diagnostic imaging related to vascular treatment, such as percutaneous coronary intervention (PCI), there is known a technology for detecting a desired object from a blood vessel tomographic image using a machine learning model. However, a blood vessel tomographic image may include an unclear image region called an artifact, and in such a case, an object may not be suitably detected.

For example, there is a known program for detecting an object region from a medical image using a machine learning model, calculating the reliability of each boundary point located on the boundary of the object region, and correcting the boundary of the object region using boundary points at which the calculated reliability satisfies a predetermined criterion.

### SUMMARY

In one aspect, embodiments of the present disclosure provide a medical device, a method, and a storage medium capable of suitably correcting the boundary of a detection target region.

In one embodiment, a medical device for processing a blood vessel tomographic image, comprises: a display; a memory that stores a program; and a processor configured to execute the program to perform the steps of: acquiring data indicating a blood vessel tomographic image, executing a call to a machine learning model with the acquired data to detect a plurality of target regions in the blood vessel tomographic image and an unclear part of the blood vessel tomographic image, the machine learning model having been trained with: a plurality of training blood vessel tomographic images, and ground truth labels indicating positions of target regions and unclear parts for the training blood vessel tomographic images, identifying a first boundary portion of a target region among the plurality of target regions, the first boundary portion being outside the detected unclear part, correcting a second boundary portion of the target region that is inside the detected unclear part, using the identified first boundary portion, generating a screen showing: the blood vessel tomographic image, and a boundary line superimposed on the blood vessel tomographic image, the boundary line including the first boundary portion and the corrected second boundary portion, and controlling the display to display the generated screen.

In one aspect, the boundary of a detection target region can be suitably corrected.

### BRIEF DESCRIPTION OF DRAWINGS

FIG. 1 is a system diagram of an image diagnosis system.

FIG. 2 is a block diagram illustrating a configuration example of a server.

FIG. 3 is a block diagram illustrating a configuration example of an image diagnosis apparatus.

FIG. 4 is an explanatory view illustrating a detection example of a detection target region.

FIG. 5 is an explanatory view related to a detection model.

FIG. 6 is an explanatory view related to an annotation process for an unclear region.

FIG. 7 is an explanatory view related to a boundary correction process.

FIG. 8 is an explanatory view illustrating a display example of a blood vessel tomographic image.

FIG. 9 is a flowchart illustrating a process of generating a detection model.

FIG. 10 is a flowchart illustrating a process of detecting a detection target region.

FIG. 11 is an explanatory view related to a detection model.

FIG. 12 is an explanatory view related to a process of assigning a label to an unclear region.

FIG. 13 is a flowchart illustrating a process of detecting a detection target region.

### DESCRIPTION OF EMBODIMENTS

Hereinafter, embodiments of the present disclosure will be described in detail based on the accompanying drawings.

(Embodiment 1)

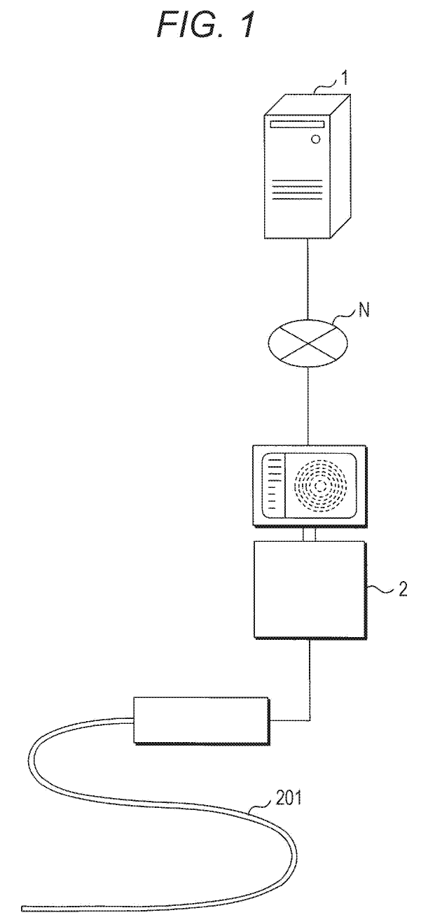

FIG. 1 is a system diagram of an image diagnosis system according to Embodiment 1. The present embodiment will describe an image diagnosis system that detects a predetermined detection target region (e.g., the lumen of a blood vessel, the inside of a stent, or the inner side of an external elastic membrane) from a blood vessel tomographic image obtained by imaging a cross-section of a blood vessel of a patient, and corrects the boundary of the detection target region. The image diagnosis system includes a server 1 and an image diagnosis apparatus 2. Such apparatuses are communicably connected via a network N.

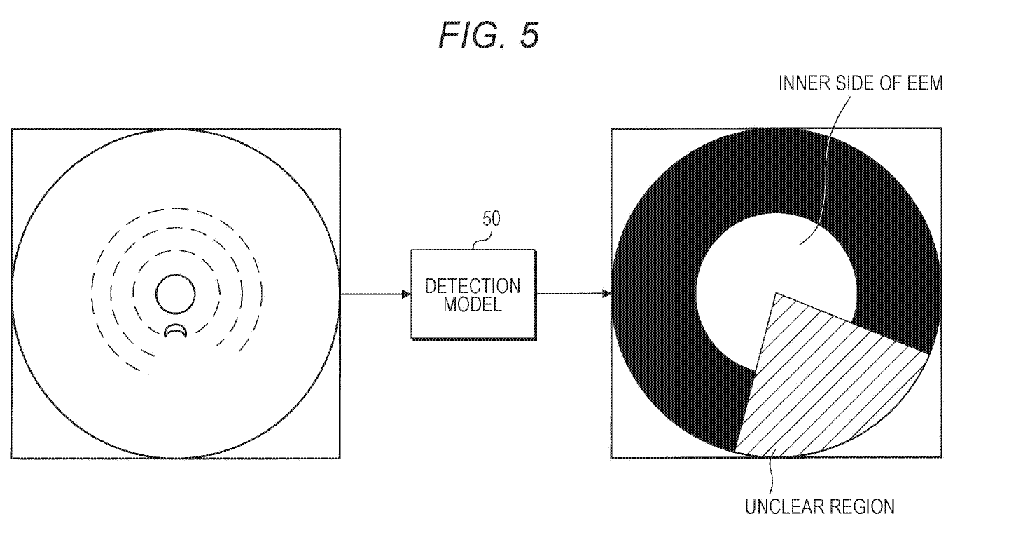

The server 1 is a server computer that can perform various types of information processing, and transmission and reception of information. Note that the apparatus corresponding to the server 1 may be a device such as a personal computer. The server 1 performs machine learning on predetermined training data to generate a detection model 50 (see FIG. 5) that detects a plurality of detection target regions including an unclear region (hereinafter also referred to as an unclear part) upon receiving a blood vessel tomographic image as an input.

The image diagnosis apparatus 2 is a medical device that generates a medical image of a blood vessel of a patient, and is, for example, an intravascular ultrasound (IVUS) apparatus that performs ultrasonic inspection using a catheter 201. The catheter 201 is a medical instrument inserted into a blood vessel of a patient and transmits an ultrasonic signal from an ultrasonic probe attached to the distal end of the catheter 201 and receives a reflected wave. The image diagnosis apparatus 2 generates an ultrasonic tomographic image based on the reflected wave received by the catheter 201 and displays the image.

Note that in the present embodiment, the image diagnosis apparatus 2 will be described as an IVUS apparatus, but it may also be an optical imaging apparatus based on optical coherence tomography (OCT), for example. Alternatively, the image diagnosis apparatus 2 may be a dual sensor system having both IVUS and OCT functions. In addition, the blood vessel tomographic image may be an image captured with a method other than IVUS or OCT.

The image diagnosis apparatus 2 has installed thereon data on the detection model 50 generated by the server 1. The image diagnosis apparatus 2 inputs into the detection model 50, a blood vessel tomographic image captured with the catheter 201, and detects a detection target region. As described later, the image diagnosis apparatus 2 detects an unclear region, which is an artifact, when detecting a detection target region, and corrects the boundary of the detection target region based on a region detected as the unclear region.

Although the present embodiment illustrates an example in which the image diagnosis apparatus 2 detects a detection target region using the detection model 50, the server 1 on the cloud may execute a process using the detection model 50. Alternatively, for example, a general-purpose computer connected to the image diagnosis apparatus 2 may perform the process. As such, a processing entity that executes a series of processes is not limited to a particular entity.

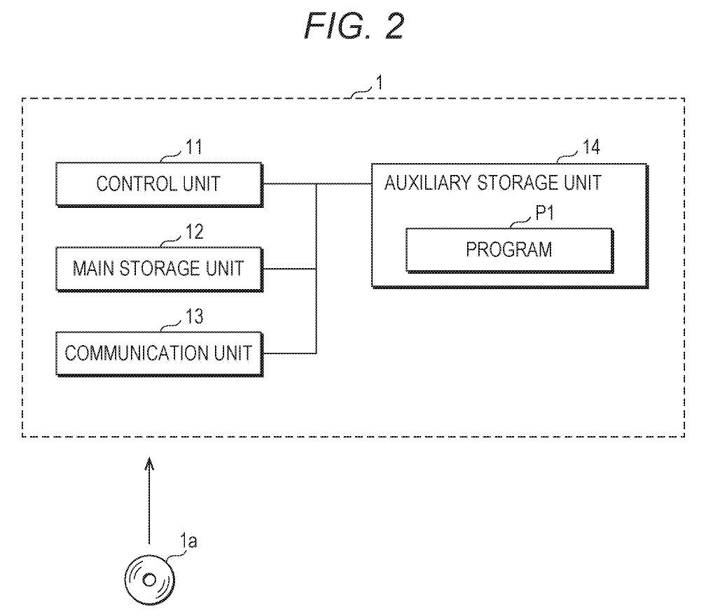

FIG. 2 is a block diagram illustrating a configuration example of the server 1. The server 1 includes a control unit 11, a main storage unit 12, a communication unit 13, and an auxiliary storage unit 14.

The control unit 11 includes one or more arithmetic processing units, such as central processing units (CPUs), micro-processing units (MPUs), and graphics processing units (GPUs), and executes various types of information processing, a control process, and the like by reading a program P1 stored in the auxiliary storage unit 14 and executing it. The main storage unit 12 is a temporary storage area, such as a static random access memory (SRAM) or a dynamic random access memory (DRAM), and temporarily stores data necessary for the control unit 11 to execute arithmetic processing. The communication unit 13 is a communication module for performing a communication-related process, and performs transmission and reception of information to/from an external device. The auxiliary storage unit 14 is a non-volatile storage area, such as a high-capacity memory or a hard disk, and stores the program P1 (i.e., a program product) necessary for the control unit 11 to execute a process, and other data.

Note that the auxiliary storage unit 14 may be an external storage device connected to the server 1. In addition, the server 1 may be a multi-computer including a plurality of computers, or may be a virtual machine constructed by software in a virtual manner.

In the present embodiment, the server 1 is not limited to the above-described configuration, and may include an input unit that receives an operation input, and a display unit that displays an image, for example. In addition, the server 1 may include a reading unit that reads a portable storage medium 1a, such as a compact disk (CD)-ROM or a digital versatile disc (DVD)-ROM, and may read the program P1 from the portable storage medium 1a and execute it.

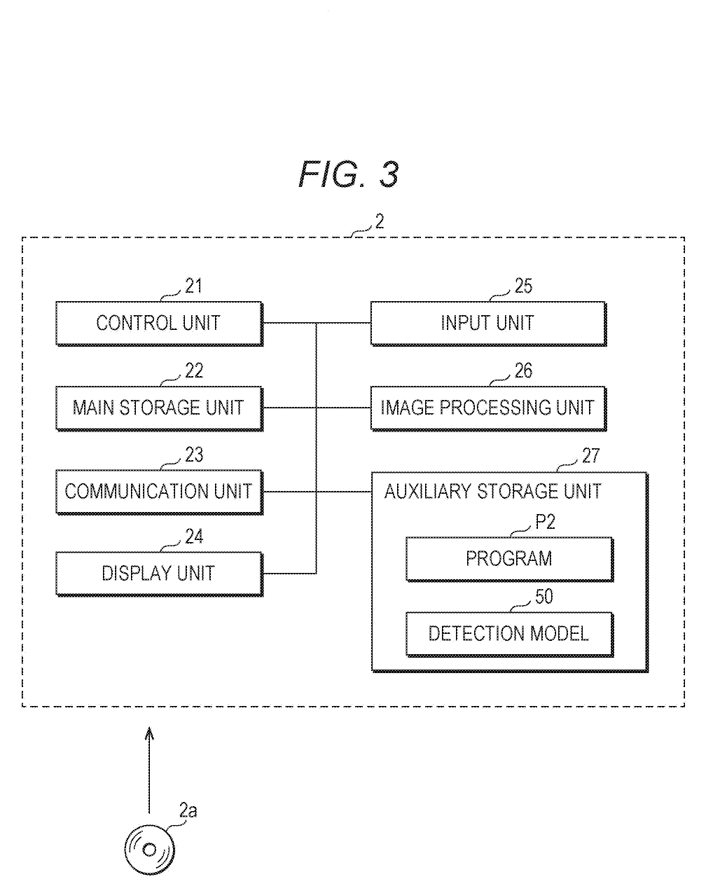

FIG. 3 is a block diagram illustrating a configuration example of the image diagnosis apparatus 2. The image diagnosis apparatus 2 includes a control unit 21, a main storage unit 22, a communication unit 23, a display unit 24, an input unit 25, an image processing unit 26, and an auxiliary storage unit 27.

The control unit 21 includes one or more arithmetic processing devices, such as CPUs, and performs various types of information processing, a control process, and the like by reading a program P2 stored in the auxiliary storage unit 27 and executing it. The main storage unit 22 is a temporary storage area, such as a RAM, and temporarily stores data necessary for the control unit 21 to execute arithmetic processing. The communication unit 23 is a communication module for performing a communication-related process, and performs transmission and reception of information to/from an external device. The display unit 24 is a display screen, such as a liquid crystal display, and displays an image. The input unit 25 is an operation interface, such as a keyboard or a mouse, and receives an operation input from a user. The image processing unit 26 is an image processing module that processes signals transmitted and received via the catheter 201, and generates a blood vessel tomographic image.

The auxiliary storage unit 27 is a non-volatile storage area, such as a hard disk or a high-capacity memory, and stores the program P2 necessary for the control unit 21 to execute a process, and other data. In addition, the auxiliary storage unit 27 stores the detection model 50. The detection model 50 is a machine learning model generated by training on predetermined training data, and is a trained model that detects a detection target region or the like upon receiving a blood vessel tomographic image generated by the image diagnosis apparatus 2 as an input.

Note that the image diagnosis apparatus 2 may include a reading unit that reads a portable storage medium 2a, such as a CD-ROM, and may read the program P2 from the portable storage medium 2a and execute it.

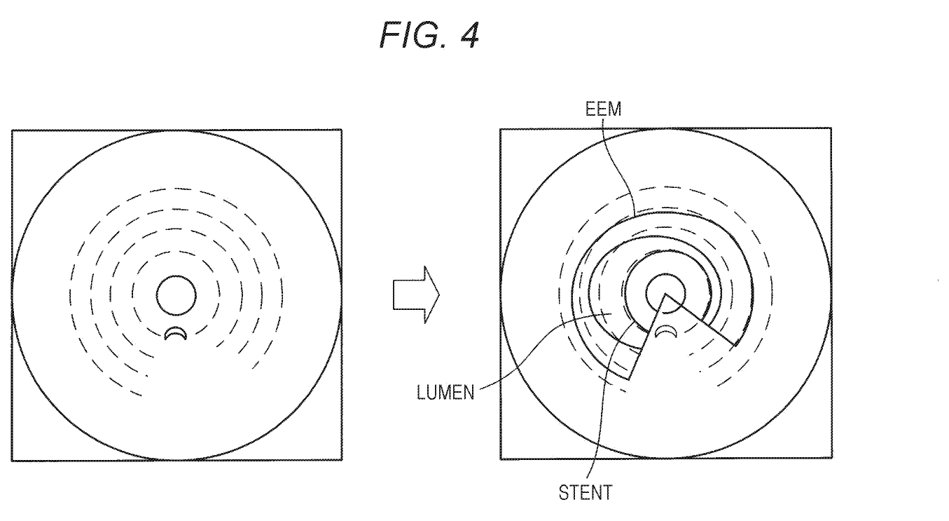

FIG. 4 is an explanatory view illustrating a detection example of a detection target region. FIG. 4 illustrates a state in which a detection target region, such as the lumen of a blood vessel, a stent, or an external elastic membrane (EEM, or an external elastic lamina (EEL)), is detected from a blood vessel tomographic image (illustrated on the left side) using a machine learning model. An unclear region that appears in the blood vessel tomographic image will be described with reference to FIG. 4.

Note that in the present embodiment, illustration and description will be made based on the assumption that a “blood vessel tomographic image” basically means a transverse tomographic image (i.e., an image obtained by imaging a cross-section of a blood vessel in a direction crossing its axial direction), but the present embodiment is not limited thereto, and a detection target may be detected from a longitudinal tomographic image of a blood vessel (i.e., an image obtained by imaging a cross-section of a blood vessel along its axial direction).

To detect a desired detection target object from a blood vessel tomographic image, it is possible to perform a process related to semantic segmentation using a machine learning model, for example. Semantic segmentation is a method of identifying a target object from an image on a pixel-by-pixel basis, and is executed using a convolutional neural network (CNN) typified by U-Net, for example.

Training a machine learning model for semantic segmentation on the features of various target objects makes it possible to detect a large number of target objects from one image. For example, as illustrated on the right side of FIG. 4, a region indicating the lumen of a blood vessel, a region indicating the inside of a stent, a region indicating the inner side of an external elastic membrane, and the like can be identified from a blood vessel tomographic image on a pixel-by-pixel basis.

Meanwhile, from the detection result on the right side of FIG. 4, it is seen that a region, such as the lumen of a blood vessel, is not circular, and its contour changes rapidly. This is because the image includes an unclear region, and the machine learning model was unable to identify such a region. The unclear region refers to a region where the boundary of the lumen of a blood vessel, the boundary of an external elastic membrane, or the like cannot be clearly identified (i.e., annotated). In the example of FIG. 4, since the image includes as an unclear region a guide wire as well as a shadow region (i.e., an acoustic shadow) of the guide wire, the model cannot identify the boundary of the lumen or the like in the unclear region. Thus, the boundary of the lumen or the like is missing in a fan shape.

As described above, when the image includes an unclear region, it is possible to simply correct (i.e., interpolate) the contour using spline interpolation or the like, but such correction can only be performed on the premise that there is no abrupt change in the contour. Thus, when the contour includes an abrupt change as illustrated in FIG. 4, the contour is difficult to correct. Therefore, in the present embodiment, not only a detection target object, such as a lumen, but also an unclear region is detected to allow for the correction of the contour (i.e., the boundary) of the detection target object.

FIG. 5 is an explanatory view related to the detection model 50. FIG. 5 illustrates a state in which a plurality of (i.e., two in FIG. 5) detection target regions including an unclear region are detected when a blood vessel tomographic image is input into the detection model 50.

The detection model 50 is a machine learning model that has been trained on predetermined training data, and is a model trained to detect a plurality of detection target regions including an unclear region upon receiving a blood vessel tomographic image as an input. For example, the detection model 50 is a model (CNN) related to semantic segmentation, such as U-Net, and detects a detection target region on a pixel-by-pixel basis.

Note that the detection model 50 may be a neural network other than the CNN. Furthermore, the detection model 50 may be a machine learning model other than the neural network, such as a decision tree or a support vector machine (SVM), for example.

Although the present embodiment illustrates an example in which a blood vessel tomographic image generated (i.e., constructed) from data on a reflected wave of ultrasound is used as an input of the detection model 50, the present embodiment is not limited thereto. It is also possible to use raw data on a reflected wave to serve as the basis of a blood vessel tomographic image as an input of the detection model 50. That is, it is acceptable as long as the detection model 50 can receive data indicating a blood vessel tomographic image as an input, and the data is not limited to image data.

Examples of the detection target regions include a region indicating the lumen (of a main trunk) of a blood vessel, a region indicating the inner side of a stent, a region indicating the inner side of an external elastic membrane (of a main trunk) of a blood vessel, a region indicating a side branch of a blood vessel, a region indicating plaque, a thrombus region, a hematoma region, a calcified region, a dissection region, a crack region, an extravascular tissue region, and a device region. Note that the “device” herein refers to a device, such as a guide wire, inserted into a blood vessel of a patient.

The above are merely examples of the detection target regions, and thus, other detection targets may be detected. The detection model 50 according to the present embodiment detects, for such a plurality of detection targets, detection target regions indicating the detection targets. In the following description, a case where an external elastic membrane (EEM) of a blood vessel is mainly detected will be illustrated and described as an example for the sake of simplicity.

The server 1 generates the detection model 50 by training on training data. The training data is data obtained by assigning a label indicating the correct position of a detection target region (i.e., a region indicating the inner side of the external elastic membrane) to a blood vessel tomographic image for training.

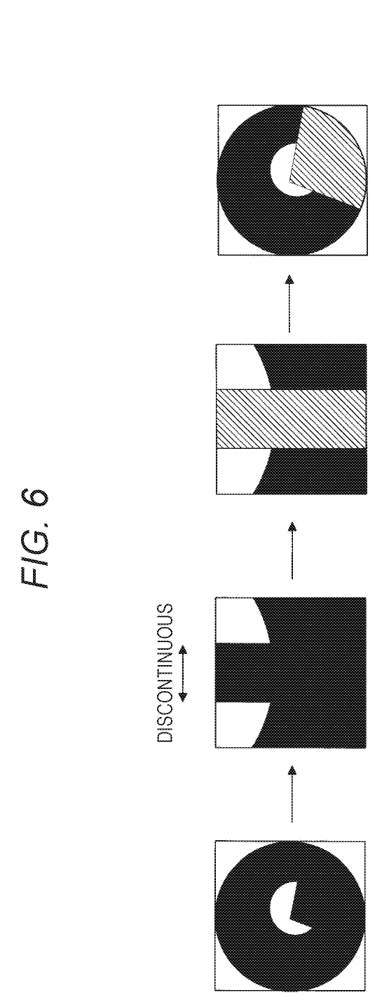

FIG. 6 is an explanatory view related to an annotation process for an unclear region. FIG. 6 illustrates a state in which a label indicating the position of an unclear region is assigned to the above-described blood vessel tomographic image for training (i.e., annotation).

When the above-described training data is provided, the server 1 assigns a label for identifying an unclear region to a blood vessel tomographic image for training. Specifically, the server 1 assigns a label indicating an unclear region when the boundary of a detection target region (i.e., a region indicating the inner side of an external elastic membrane) assigned with a label in advance is discontinuous or when the boundary of the detection target region does not have a specific shape (e.g., a circular shape).

For example, first, the server 1 converts a blood vessel tomographic image in an orthogonal coordinate system (i.e., X and Y coordinates) into an image in a polar coordinate system (i.e., r and θ coordinates) in which the vertical axis represents the distance from an ultrasonic probe and the horizontal axis represents an angle. Based on the polar coordinate image obtained through conversion, the server 1 determines whether or not a detection target region (i.e., a hollow region in FIG. 6) is discontinuous.

If it is determined that the detection target region is discontinuous, the server 1 determines that the discontinuous portion is an unclear region. In such a case, the server 1 assigns a label (illustrated by hatching in FIG. 6) indicating the unclear region to the discontinuous portion. The server 1 converts the polar coordinate image assigned with the label into an orthogonal coordinate image to obtain a blood vessel tomographic image for training.

Described above is a case where an unclear region is determined from a polar coordinate image. However, for example, the server 1 may determine whether a detection target region is circular based on an orthogonal coordinate image, and determine, when it is determined that the detection target region is not circular, a portion that is not circular (i.e., a portion missing in a fan shape) as an unclear region. As described above, various methods may be used as a method of assigning a label to an unclear region.

Referring again to FIG. 5, the description will be continued. After assigning a label indicating an unclear region to a blood vessel tomographic image for training, the server 1 generates the detection model 50 based on the training data. That is, the server 1 detects each detection target region including an unclear region by inputting a blood vessel tomographic image for training into the detection model 50. The server 1 compares the detected detection target region with a correct label, and updates a parameter, such as a weight between neurons, so that the two become closer to each other. The server 1 sequentially inputs blood vessel tomographic images for training to the detection model 50 to update the parameter, and finally generates (i.e., constructs) the detection model 50 with the optimized parameter. Data on the generated detection model 50 is installed on the image diagnosis apparatus 2.

When a blood vessel tomographic image of a patient is actually captured and a detection target region is detected from the captured blood vessel tomographic image, the image diagnosis apparatus 2 inputs the captured blood vessel tomographic image into the detection model 50 to detect a plurality of detection target regions including an unclear region. Specifically, as indicated by a hollow portion (i.e., the inner side of the external elastic membrane) or hatching (i.e., an unclear region) on the right side of FIG. 5, the image diagnosis apparatus 2 assigns a label indicating which detection target region each pixel corresponds to and a class of the detection target region.

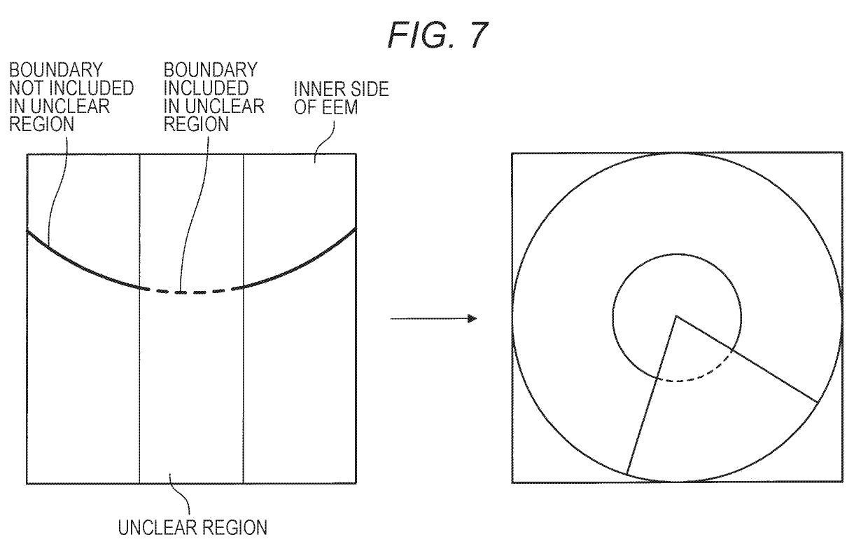

FIG. 7 is an explanatory view related to a boundary correction process. FIG. 7 illustrates a state in which the boundary of another detection target region is corrected (i.e., interpolated) based on an unclear region. In FIG. 7, hatching or black shading is omitted for convenience of illustration.

Upon detecting a detection target region including an unclear region, the image diagnosis apparatus 2 corrects, based on a region detected as the unclear region, the boundary of another detection target region other than the unclear region. For example, as illustrated on the left side of FIG. 7, first, the image diagnosis apparatus 2 converts an orthogonal coordinate image, from which a detection target region has been detected, into a polar coordinate image. Although FIG. 7 illustrates an example in which the boundary of a detection target region is corrected based on a polar coordinate image, the present embodiment is not limited thereto, and the boundary may be corrected based on an orthogonal coordinate image.

From a blood vessel tomographic image (i.e., a polar coordinate image), the image diagnosis apparatus 2 identifies a boundary portion not included in an unclear region, at the boundary of another detection target region (i.e., on the inner side of the EEM) other than the unclear region. Specifically, the image diagnosis apparatus 2 identifies a boundary portion not in contact with the unclear region. In FIG. 7, such a boundary portion is indicated by a thick line.

Based on the boundary portion identified as above, the image diagnosis apparatus 2 corrects (i.e., interpolates) a boundary portion included in the unclear region with reference to another detection target region other than the unclear region. Specifically, the image diagnosis apparatus 2 generates a new boundary line using spline interpolation based on the boundary line indicated by the thick line in FIG. 7. In FIG. 7, the newly generated boundary line is indicated by a thick dotted line. As described above, detecting an unclear region makes it possible to identify a boundary portion (i.e., a boundary portion not included in the unclear region) to be used as the basis for correction (i.e., interpolation), and thus appropriately correct a boundary portion included in the unclear region. The image diagnosis apparatus 2 converts the polar coordinate image into an orthogonal coordinate image, and displays a blood vessel tomographic image with the corrected boundary of the detection target region.

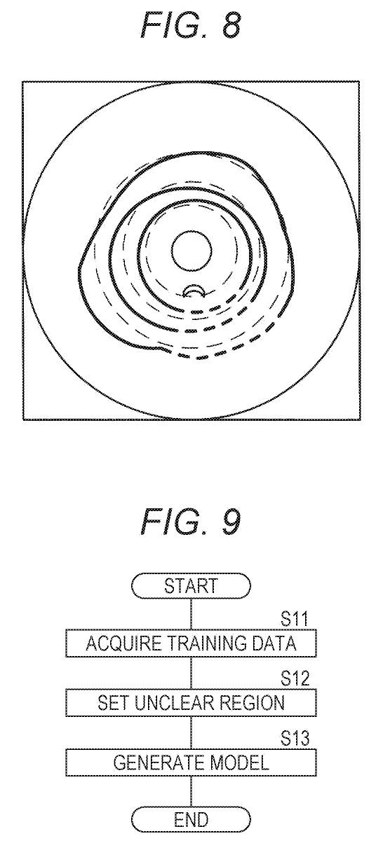

FIG. 8 is an explanatory view illustrating a display example of the blood vessel tomographic image. For example, the image diagnosis apparatus 2 displays the blood vessel tomographic image on which a boundary line indicating the boundary of each detection target region (e.g., the lumen of a blood vessel, a stent, and an external elastic membrane in FIG. 8) is superimposed. In such a case, the image diagnosis apparatus 2 displays the corrected boundary portion in a display style different from that of the uncorrected boundary portion. Specifically, as illustrated in FIG. 8, the image diagnosis apparatus 2 displays the corrected boundary portion using a dotted line. Accordingly, it is possible to indicate a user (i.e., a medical professional) which part has been corrected.

Although FIG. 8 illustrates an example in which boundary lines for only the detection target regions other than the unclear region are superimposed, it is also possible to clearly indicate the unclear region to the user by also superimposing a boundary line for the unclear region.

FIG. 9 is a flowchart illustrating a process of generating the detection model 50. A process performed to generate the detection model 50 using machine learning will be described with reference to FIG. 9.

The control unit 11 of the server 1 acquires training data for generating the detection model 50 (S11). The training data is data obtained by assigning a label indicating the correct position of a detection target region to a blood vessel tomographic image for training. Examples of the detection target region include a region indicating the lumen of a blood vessel, a region indicating the inside of a stent, and a region indicating the inner side of an external elastic membrane.

If the boundary of the detection target region indicated by the correct label (i.e., the ground truth label) does not satisfy a predetermined condition, the control unit 11 assigns a label indicating an unclear region to the blood vessel tomographic image for training (S12). Specifically, the control unit 11 assigns a label indicating an unclear region when the boundary of a detection target region in a blood vessel tomographic image (i.e., a polar coordinate image) is discontinuous or when the boundary of a detection target region in a blood vessel tomographic image (i.e., an orthogonal coordinate image) is not circular, for example.

Based on the training data, the control unit 11 generates the detection model 50 trained to detect a plurality of detection target regions including an unclear region upon receiving a blood vessel tomographic image as an input (S13). For example, the control unit 11 generates a CNN related to semantic segmentation as the detection model 50. The control unit 11 detects a detection target region including an unclear region by inputting a blood vessel tomographic image for training into the detection model 50. The control unit 11 compares the detected detection target region with a correct label, and optimizes a parameter, such as a weight between neurons, so that the two become closer to each other, thereby generating the detection model 50. The control unit 11 ends the series of processes.

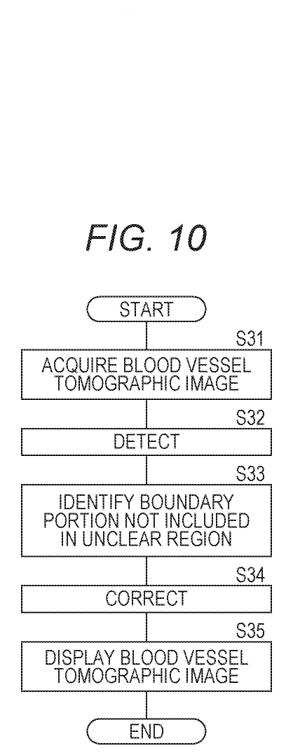

FIG. 10 is a flowchart illustrating a process of detecting a detection target region. A process performed to detect a detection target region using the detection model 50, and correct the boundary of the detection target region will be described with reference to FIG. 10.

The control unit 21 of the image diagnosis apparatus 2 acquires a blood vessel tomographic image obtained by imaging a cross-section of a blood vessel of a patient (S31). For example, the control unit 21 acquires a transverse tomographic image obtained by imaging a cross-section of a blood vessel of a patient in a direction crossing (i.e., orthogonal to) its axial direction. The control unit 21 detects a plurality of detection target regions including an unclear region by inputting the acquired blood vessel tomographic image into the detection model 50 (S32).

The control unit 21 identifies, from the boundary of another detection target region other than the unclear region, a boundary portion not included in the unclear region (S33). That is, the control unit 21 identifies a boundary portion of a detection target region not in contact with the unclear region. Based on the identified boundary portion, the control unit 21 corrects a boundary portion included in the unclear region, at the boundary of the other detection target region (S34). For example, the control unit 21 corrects (i.e., interpolates) the boundary portion included in the unclear region based on the boundary portion not included in the unclear region, using spline interpolation or the like as described above.

The control unit 21 displays a blood vessel tomographic image indicating the detection target region with the corrected boundary (S35). For example, the control unit 21 displays a blood vessel tomographic image on which a boundary line for each detection target region is superimposed. In such a case, the control unit 21 displays the corrected boundary portion in a display style different from that of the uncorrected boundary portion. The control unit 21 ends the series of processes.

The above-described configuration provides a specific technical solution to a problem inherently rooted in computer-based medical image processing. Conventionally, when a blood vessel tomographic image includes an artifact such as an acoustic shadow cast by a guide wire, the boundary of a target object (e.g., a lumen or an external elastic membrane) changes abruptly or becomes missing, making standard contour interpolation techniques ineffective. The present embodiment improves the functioning of the image processing apparatus itself by employing a trained machine learning model to explicitly detect the unclear region alongside the target regions. By computationally determining exactly which part of the image constitutes the unclear region, the system can reliably isolate a first boundary portion located outside the unclear region and use it as an accurate algorithmic basis to correct a second boundary portion located inside the unclear region. This specific rules-based approach enables the medical device to accurately reconstruct missing anatomical boundaries even in the presence of severe imaging artifacts, thereby generating a technically improved, continuous boundary line and providing a visually enhanced user interface on the display.

(Embodiment 2)

Embodiment 1 has described a configuration in which multi-class classification (i.e., detection) is performed, that is, determining to which detection target region each pixel in a blood vessel tomographic image belongs. The present embodiment will describe a configuration in which multiple labels are assigned, that is, a plurality of labels indicating a plurality of detection target regions are assigned to a pixel in which the plurality of detection target regions overlap (i.e., an overlapping region). Note that the same components as those in Embodiment 1 are denoted by the same reference numerals, and the description thereof will be omitted.

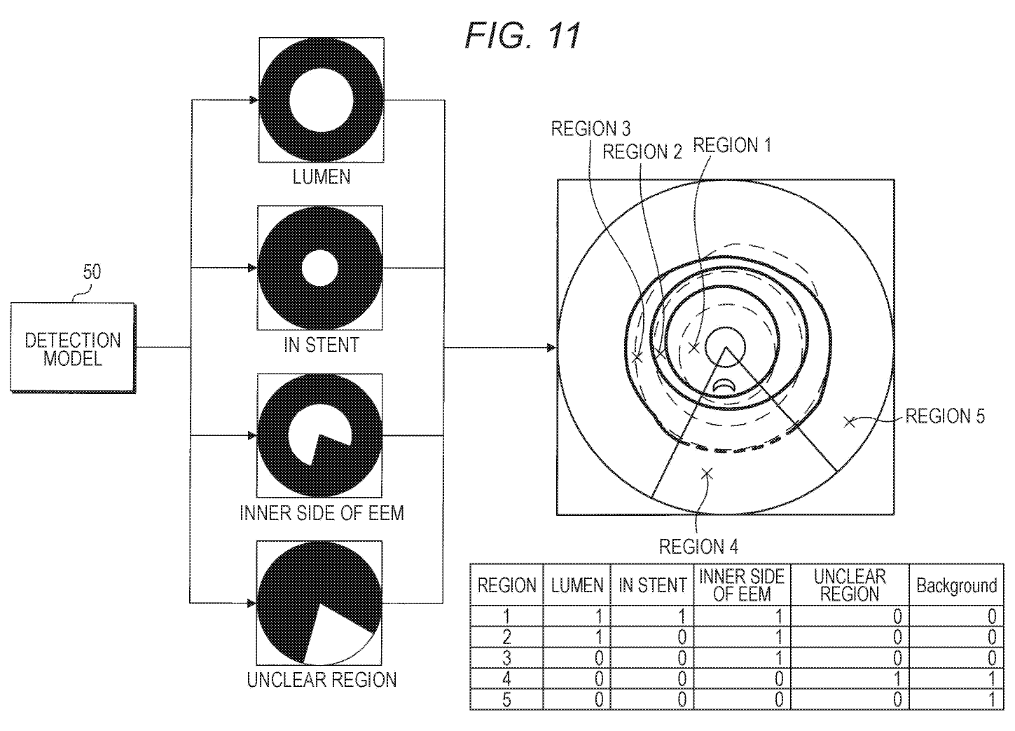

FIG. 11 is an explanatory view related to the detection model 50 according to Embodiment 2. FIG. 11 illustrates a state in which the detection model 50 outputs label data for each type of detection target region (i.e., a lumen, a stent, an external elastic membrane (EEM), and an unclear region), and the plurality of pieces of label data are superimposed so that a blood vessel tomographic image on which the boundary lines of the detection target regions are superimposed is output.

The detection model 50 according to Embodiment 1 performs multi-class classification, that is, determination of which type of detection target region each pixel in a blood vessel tomographic image belongs to. Meanwhile, the detection model 50 according to the present embodiment performs two-class classification for each type of detection target region, and the classification results of the respective types are superimposed, thereby enabling the identification of the boundary of each detection target region.

Specifically, the detection model 50 according to the present embodiment is a model trained to, upon receiving a blood vessel tomographic image as an input, assign a plurality of labels indicating each of a plurality of detection target regions to an overlapping region where the plurality of detection target regions overlap, and assign one label indicating a detection target region to a non-overlapping region where the detection target region does not overlap other detection target regions. By inputting a blood vessel tomographic image into the detection model 50, the image diagnosis apparatus 2 assigns a plurality of labels to an overlapping region, and assigns one label to a non-overlapping region.

That is, as illustrated on the left side of FIG. 11, the detection model 50 according to the present embodiment generates, for each type of detection target region, a plurality of types of label data (i.e., label images) indicating whether or not each pixel belongs to the detection target region using a binary value of “1” or “0”. In the example of FIG. 11, the detection model 50 generates four types of label data including label data representing a region indicating the lumen of a blood vessel, label data representing a region indicating the inside of a stent, label data representing a region indicating the inner side of an external elastic membrane, and label data representing an unclear region. The image diagnosis apparatus 2 generates different label data according to the type of each detection target region by inputting a blood vessel tomographic image into the detection model 50.

The image diagnosis apparatus 2 identifies the respective regions in the blood vessel tomographic image by superimposing the pieces of label data output from the detection model 50 one on top of the others. This will be described with reference to a blood vessel tomographic image on the right side of FIG. 11 and a table on the lower right side of FIG. 11. For example, when considering a point (i.e., a pixel) of a “Region 1” in the blood vessel tomographic image, it is found by referring to each piece of label data that a value (i.e., the label) indicating whether the point corresponds to a lumen is “1”, a value indicating whether or not the point is inside a stent is “1”, a value indicating whether or not the point is on the inner side of an external elastic membrane is “1”, a value indicating whether or not the point corresponds to an unclear region is “0”, and a value indicating whether or not the point corresponds to other regions (“Background”) is “0”. That is, it is found that the "Region 1" is located in the lumen of a blood vessel, in a stent, and on the inner side of an external elastic membrane, but is not an unclear region.

Likewise, when considering a “Region 2”, it is found that the value of “Lumen” is “1”, the value of “In Stent” is “0”, the value of “Inner Side of EEM” is “1”, the value of “Unclear Region” is “0”, and the value of “Background” is “0”. In such a case, it is found that the "Region 2" is located in the lumen of a blood vessel and on the inner side of an external elastic membrane, but is not located in a stent and is not an unclear region.

Note that the detection model 50 may separately detect “Background” corresponding to each detection target, such as “Background” of “Lumen”, “Background” of “In Stent”, and “Background” of “Inner Side of EEM”.

As described above, the detection model 50 according to the present embodiment generates, for each type of detection target, a plurality of pieces of label data indicating whether or not each pixel belongs to the detection target region based on two-class classification. In contrast, the detection model 50 according to Embodiment 1 simply generates label data indicating which type of detection target region each pixel belongs to based on multi-class classification. Referring to the example on the right side of FIG. 11, the detection model 50 according to Embodiment 1 assigns a multi-class label, for example, assigns a label of “1” indicating a lumen for the “Region 1”, assigns a label of “2” indicating the inside of a stent for the “Region 2”, assigns a label of “3” indicating the inner side of an external elastic membrane for the “Region 3”, and the like.

When multi-class semantic segmentation is performed as in Embodiment 1, a task to be executed by the detection model 50 tends to be complex because it performs multi-class classification. Therefore, as the number of detection target regions to be detected increases and the division of a region in an image becomes more complex, the individual detection accuracy tends to decrease, and also, the training cost (i.e., training amount) of the detection model 50 tends to increase for each segment obtained by dividing the region.

In contrast, since the detection model 50 according to the present embodiment only needs to perform two-class classification for each type of detection target region, it is possible to suppress an increase in the training cost of the detection model 50 while preventing a decrease in the detection accuracy.

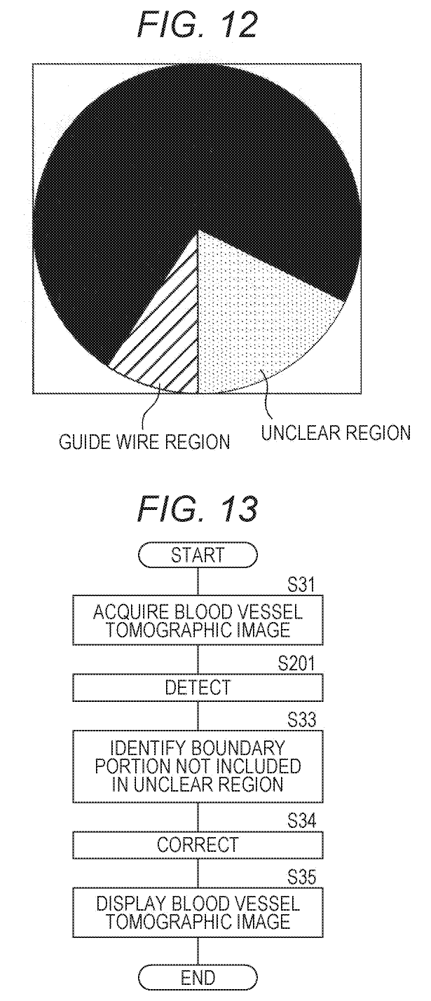

FIG. 12 is an explanatory view related to a process of assigning a label to an unclear region. FIG. 12 illustrates a state in which a label is assigned to a region, such as a guide wire, in addition to an unclear region.

The detection target regions to be detected in the present embodiment further include a region indicating a guide wire and a shadow region (i.e., an acoustic shadow) of the guide wire (hereinafter referred to as a "guide wire region" for simplicity), a region indicating a stent strut and a shadow region of the stent strut (hereinafter referred to as a "stent strut region"), and a region indicating plaque (hereinafter referred to as a "plaque region"). The guide wire region, the stent strut region, and the plaque region are regions observed as shadow portions in a blood vessel tomographic image, and are regions that can be said to be representative of unclear regions.

Note that an unclear region that is present due to an acoustic shadow of a guide wire region or the like is only one example, and the unclear region may be present due to reasons other than acoustic shadows.

The detection model 50 according to the present embodiment detects a guide wire region, a stent strut region, and a plaque region as detection target regions from a blood vessel tomographic image, separately from an unclear region. Herein, if an unclear region detected by the detection model 50 includes a region overlapping any of the guide wire region, the stent strut region, and the plaque region, the image diagnosis apparatus 2 preferentially identifies, for the overlapping region, the guide wire region, the stent strut region, or the plaque region based on its label. That is, the image diagnosis apparatus 2 detects a region of an unclear region overlapping the guide wire region, the stent strut region, or the plaque region as the guide wire region, the stent strut region, or the plaque region. Accordingly, an unclear region can be more clearly identified.

FIG. 13 is a flowchart illustrating a process of detecting a detection target region according to Embodiment 2. After acquiring a blood vessel tomographic image (S31), the image diagnosis apparatus 2 executes the following process.

The control unit 21 of the image diagnosis apparatus 2 detects a plurality of detection target regions including an unclear region by inputting the acquired blood vessel tomographic image into the detection model 50 (S201). Herein, the detection model 50 according to the present embodiment is a model trained to, upon receiving a blood vessel tomographic image as an input, assign a plurality of labels indicating each of a plurality of detection target regions to an overlapping region where the plurality of detection target regions overlap, and assign one label indicating a detection target region to a non-overlapping region where the detection target region does not overlap other detection target regions.

By inputting the acquired blood vessel tomographic image into the detection model 50, the control unit 21 assigns a plurality of labels to an overlapping region, and assigns one label to a non-overlapping region. That is, the control unit 21 generates different label data (i.e., a label image) according to the type of each detection target region. In such a case, if an unclear region detected by the detection model 50 includes a region overlapping any of the guide wire region, the stent strut region, and the plaque region, the control unit 21 preferentially assigns, for the overlapping region, a label corresponding to the guide wire region, the stent strut region, or the plaque region. The control unit 21 proceeds to S33.

As described above, according to Embodiment 2, it is possible to suppress an increase in the training cost of the detection model 50 while preventing a decrease in the detection accuracy for detection target regions.

It should be understood that the embodiments disclosed herein are illustrative in all respects and not restrictive. The scope of the present disclosure is indicated not by the forgoing description but by the claims, and is intended to include all changes that fall within the meaning and scope equivalent to the claims.

Any or all of the features described in the respective embodiments can be combined. In addition, the independent claims and their dependent claims can be combined, regardless of their dependent relationships. Furthermore, although a format in which a claim is dependent on two or more other claims (multiple dependent claim format) is used in the claims, the claim form is not limited to this. The claims may be described in a format in which a multiple dependent claim is dependent on at least one other multiple dependent claim (multiple multiple dependent claim).

What is claimed is:

1. A medical device for processing a blood vessel tomographic image, comprising:
a display;
a memory that stores a program; and
a processor configured to execute the program to perform the steps of:
acquiring data indicating a blood vessel tomographic image,
executing a call to a machine learning model with the acquired data to detect a plurality of target regions in the blood vessel tomographic image and an unclear part of the blood vessel tomographic image, the machine learning model having been trained with:
a plurality of training blood vessel tomographic images, and
ground truth labels indicating positions of target regions and unclear parts for the training blood vessel tomographic images,
identifying a first boundary portion of a target region among the plurality of target regions, the first boundary portion being outside the detected unclear part,
correcting a second boundary portion of the target region that is inside the detected unclear part, using the identified first boundary portion,
generating a screen showing:
the blood vessel tomographic image, and
a boundary line superimposed on the blood vessel tomographic image, the boundary line including the first boundary portion and the corrected second boundary portion, and
controlling the display to display the generated screen.

2. The medical device according to claim 1, wherein the target regions include at least one of a region indicating a lumen of a blood vessel, a region indicating an inside of a stent, and a region indicating an inner side of an external elastic membrane.

3. The medical device according to claim 1, wherein
the machine learning model has been trained to, when data indicating a blood vessel tomographic image is received as an input, assign a plurality of labels respectively indicating a plurality of target regions to an overlapping region where the plurality of target regions overlap, and assign a single label indicating one target region to a non-overlapping region where the one target region does not overlap other target regions, and
executing the call includes assigning the plurality of labels to the overlapping region and assigning the single label to the non-overlapping region.

4. The medical device according to claim 3, wherein
the target regions further include at least one of a region indicating a guide wire and a shadow region of the guide wire, a region indicating a stent strut and a shadow region of the stent strut, and a region indicating plaque, and
the steps further comprise, when the detected unclear part includes a region overlapping any of the region indicating the guide wire and the shadow region of the guide wire, the region indicating the stent strut and the shadow region of the stent strut, and the region indicating plaque, detecting the overlapping region as the region indicating the guide wire and the shadow region of the guide wire, the region indicating the stent strut and the shadow region of the stent strut, or the region indicating plaque.

5. The medical device according to claim 1, wherein the generated screen displays the corrected second boundary portion in a display style different from a display style of the first boundary portion.

6. The medical device according to claim 5, wherein the generated screen displays the corrected second boundary portion as a dotted line.

7. The medical device according to claim 1, wherein the second boundary portion is corrected using spline interpolation based on the identified first boundary portion.

8. The medical device according to claim 1, wherein the steps further comprise:
converting the blood vessel tomographic image from an orthogonal coordinate system into a polar coordinate system before identifying the first boundary portion; and
converting the blood vessel tomographic image back to the orthogonal coordinate system after correcting the second boundary portion.

9. The medical device according to claim 1, wherein the blood vessel tomographic image is an intravascular ultrasound image or an optical coherence tomography image.

10. The medical device according to claim 1, wherein the ground truth labels correspond to portions where a boundary of a target region in the training blood vessel tomographic images is discontinuous or lacks a circular shape.

11. A method for processing a blood vessel tomographic image, the method comprising:
acquiring data indicating a blood vessel tomographic image;
executing a call to a machine learning model with the acquired data to detect a plurality of target regions in the blood vessel tomographic image and an unclear part of the blood vessel tomographic image, the machine learning model having been trained with:
a plurality of training blood vessel tomographic images, and
ground truth labels indicating positions of target regions and unclear parts for the training blood vessel tomographic images;
identifying a first boundary portion of a target region among the plurality of target regions, the first boundary portion being outside the detected unclear part;
correcting a second boundary portion of the target region that is inside the detected unclear part, using the identified first boundary portion; and
displaying a screen showing:
the blood vessel tomographic image, and
a boundary line superimposed on the blood vessel tomographic image, the boundary line including the first boundary portion and the corrected second boundary portion.

12. The method according to claim 11, wherein the target regions include at least one of a region indicating a lumen of a blood vessel, a region indicating an interior of a stent, and a region indicating an inner side of an external elastic membrane.

13. The method according to claim 11, wherein
the machine learning model has been trained to, when data indicating a blood vessel tomographic image is received as an input, assign a plurality of labels respectively indicating a plurality of target regions to an overlapping region where the plurality of target regions overlap, and assign a single label indicating one target region to a non-overlapping region where the one target region does not overlap other target regions, and
executing the call includes assigning the plurality of labels to the overlapping region and assigning the single label to the non-overlapping region.

14. The method according to claim 13, wherein
the target regions further include at least one of a region indicating a guide wire and a shadow region of the guide wire, a region indicating a stent strut and a shadow region of the stent strut, and a region indicating plaque, and
the method further comprises, when the detected unclear part includes a region overlapping any of the region indicating the guide wire and the shadow region of the guide wire, the region indicating the stent strut and the shadow region of the stent strut, and the region indicating plaque, detecting the overlapping region as the region indicating the guide wire and the shadow region of the guide wire, the region indicating the stent strut and the shadow region of the stent strut, or the region indicating plaque.

15. The method according to claim 11, wherein the corrected second boundary portion is displayed in a display style different from a display style of the first boundary portion.

16. The method according to claim 15, wherein the corrected second boundary portion is displayed as a dotted line.

17. The method according to claim 11, wherein the second boundary portion is corrected using spline interpolation based on the identified first boundary portion.

18. The method according to claim 11, further comprising:
converting the blood vessel tomographic image from an orthogonal coordinate system into a polar coordinate system before identifying the first boundary portion; and
converting the blood vessel tomographic image back to the orthogonal coordinate system after correcting the second boundary portion.

19. The method according to claim 11, wherein the blood vessel tomographic image is an intravascular ultrasound image or an optical coherence tomography image.

20. A non-transitory computer-readable storage medium storing a program that, when executed by a processor of a computer to perform a process comprising:
acquiring data indicating a blood vessel tomographic image;
executing a call to a machine learning model with the acquired data to detect a plurality of target regions in the blood vessel tomographic image and an unclear part of the blood vessel tomographic image, the machine learning model having been trained with:
a plurality of training blood vessel tomographic images, and
ground truth labels indicating positions of target regions and unclear parts for the training blood vessel tomographic images;
identifying a first boundary portion of a target region among the plurality of target regions, the first boundary portion being outside the detected unclear part;
correcting a second boundary portion of the target region that is inside the detected unclear part, using the identified first boundary portion; and
displaying a screen showing:
the blood vessel tomographic image, and
a boundary line superimposed on the blood vessel tomographic image, the boundary line including the first boundary portion and the corrected second boundary portion.
